(19) United States
(12) Patent Application Publication
(10) Pub. No.: US 2026/0294948 A1
(43) Pub. Date: Oct. 1, 2026

(54) RESPONSE-BASED DOSING FOR TREATING CANCER WITH A HYPOMETHYLATING AGENT AND CEDAZURIDINE

(71) Applicant: TAIHO PHARMACEUTICAL CO., LTD., Tokyo (JP)

(72) Inventors: Harold Keer, Pleasanton, CA (US); Aram Oganesian, Pleasanton, CA (US); Kim-Hien Dao, Pleasanton, CA (US)

(21) Appl. No.: 19/479,851

(22) PCT Filed: May 1, 2024
(86) PCT No.: PCT/US2024/027160

§ 371 Date: Oct. 29, 2025

Related U.S. Application Data
(60) Provisional application No. 63/499,604, filed on May 2, 2023.

(51) Int. Cl. A61K 31/7068; A61K 31/706; A61K 45/06

(52) U.S. Cl. CPC A61K 31/7068; A61K 31/706; A61K 45/06

(57) ABSTRACT

Provided according to some embodiments of the invention are methods of treating cancer in a subject in need thereof that include administering to the subject a first amount of a hypomethylating agent (e.g., decitabine) and a first amount of cedazuridine in a first dosing cycle; measuring a response of the cancer to the first dosing cycle; and administering a second, reduced amount of the hypomethylating agent and a second, reduced amount of cedazuridine in subsequent dosing cycle(s) if the response of the cancer to the first dosing cycle meets a predetermined criterion.

### STATEMENT OF PRIORITY

This application claims the benefit of U.S. Provisional Application Ser. No. 63/499,604, filed May 2, 2023, the entire contents of which are incorporated by reference herein.

### FIELD OF THE INVENTION

This invention relates to methods of treating cancer with a hypomethylating agent and cedazuridine. In particular, the invention relates to dosing regimens for treating cancer with decitabine and cedazuridine.

### BACKGROUND OF THE INVENTION

The enzymes adenosine deaminase (ADA, EC 3.5.4.4) and cytidine deaminase (CDA, EC 3.5.4.5) function to deaminate natural aminopurine and aminopyrimidine nucleosides, respectively, in human and other organisms. They may also convert active nucleoside-based drugs into inactive metabolites. CDA is a component of the pyrimidine salvage pathway. It converts cytidine and deoxycytidine to uridine and deoxyuridine, respectively, by hydrolytic deamination (Arch. Biochem. Biophys. 1991, 290, 285-292; Methods Enzymol. 1978, 51, 401-407; Biochem. J. 1967, 104, 7P). It also deaminates a number of synthetic cytosine analogs which are clinically useful drugs (Cancer Chemother. Pharmacol. 1998, 42, 373-378; Cancer Res. 1989, 49, 3015-3019; Antiviral Chem. Chemother. 1990, 1, 255-262). Conversion of the cytosine compounds to the uridine derivatives usually confers loss of therapeutic activity or addition of side-effects. It has also been shown that cancers that acquire resistance to cytosine analog drugs often overexpress CDA (Leuk. Res. 1990, 14, 751-754). Leukemic cells expressing a high level of CDA can manifest resistance to cytosine antimetabolites and thereby limit the antineoplastic activity of such therapeutics (Biochem. Pharmacol. 1993, 45, 1857-1861). CDA is also highly expressed in the gut and liver and so may affect the bioavailability of therapeutic cytidine analogs.

Decitabine (5-aza-2′-deoxycytidine), a cytidine analog, is an antineoplastic agent and hypomethylating agent (HMA) for the treatment of myelodysplastic syndromes (MDS), with potential utility for the treatment of acute myelogenous leukemia and chronic myelogenous leukemia as well.

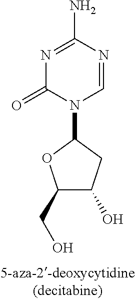

Cedazuridine ((4R)-2′-deoxy-2′,2′-difluoro-3,4,5,6-tetrahydrouridine; also known as E7727) is a CDA inhibitor. Cedazuridine and methods of making and/or using thereof are further disclosed in U.S. Pat. Nos. 8,268,800 and 9,834,576, the contents of which are incorporated by reference herein in their entirety.

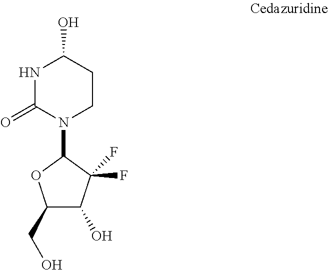

Astex Pharmaceuticals, Inc. received FDA approval for a fixed dose combination of decitabine and cedazuridine, sold under the brand name INQOVI® by Taiho Oncology, Inc. Administration of the fixed dose oral combination of decitabine and cedazuridine is typically performed in 28 day cycles, with the decitabine and cedazuridine adminstered on days 1-5 of each cycle. The dosage administered in each cycle is typically based on the expected pharmacokinetics of each compound in a subject and its observed toxicity profile. However, in some cases, such pharmacokinetic information may not be the best means of determining the correct dosage to administer in a subsequent cycle. As such, other methods of determining dosages of decitabine and cedazuridine for treating cancer may be desirable.

### SUMMARY

The inventors of the present application unexpectedly discovered that the dosage needed in a second dosing cycle for decitabine and cedazuridine may be decreased based on a response to the cancer in the first dosing cycle, as it appears that as the cancer is treated, the efficacy of a particular dose increases. For example, without wishing to be bound by any theory, is believed that hematopoietic stem cells, both malignant and non-malignant, provide a pharmacokinetic reservoir for hypomethylating agents and this reservoir of hematopoietic cells is decreased after a first dosing cycle with a hypomethylating agent such as decitabine. Thus, the efficacy of a particular dose is increased, and the dose administered in subsequent cycles may be decreased. As an additional non-limiting theory, blood cells are a major source of cytidine deaminase (CDA), and so by reducing the level of blood cells during the first dosing cycle, the overall level of CDA in the subject also decreases. As cedazuridine is a CDA inhibitor, reducing the level of CDA in a subject may allow for more cedazuridine to be available as an active agent. Therefore, a reduced amount of cedazuridine may be administered to the subject per cycle (e.g., reduced number of days of dosing) to provide a sufficient therapeutic effect. Moreover, as CDA is inhibited, it may result in an increase in available decitabine activity that may allow for a lower amount of decitabine needed for an effective dose.

Accordingly, provided according to some embodiments of the invention are methods of treating cancer in a subject in need thereof that include administering to the subject a first amount of decitabine (and/or another hypomethylating agent) and a first amount of cedazuridine in a first dosing cycle; measuring a response of the cancer to the first dosing cycle; and administering a second, reduced amount of decitabine (and/or another hypomethylating agent) and a second, reduced amount of cedazuridine in subsequent dosing cycle(s) if the response of the cancer to the first dosing cycle meets a predetermined criterion.

In some embodiments of the invention, the first dosing cycle and the subsequent dosing cycle(s) have a length of about 25 to about 30 days (e.g., 28 days). In some embodiments, in the first dosing cycle, the decitabine (and/or another hypomethylating agent) and cedazuridine are administered for about 3 to about 7 days (e.g., 5 days). In some embodiments, in subsequent dosing cycle(s), the decitabine (and/or another hypomethylating agent) and cedazuridine are administered for fewer days per cycle, such as, for example, 1-6 days (e.g., 3 days). In some embodiments, the decitabine (and/or another hypomethylating agent) and cedazuridine are administered for the same number of days in the first dosing cycle and the subsequent dosing cycle(s) but the dose administered per day is less in the subsequent dosing cycle(s) than in the first dosing cycle.

In some embodiments of the invention, the decitabine (and/or another hypomethylating agent) and the cedazuridine are administered intravenously, orally, and/or subcutaneously. In certain embodiments, the decitabine (and/or other hypomethylating agent) and cedazuridine are administered in a single solid oral dosage form in both the first dosing cycle and in subsequent dosing cycle(s).

In some embodiments, the first amount of decitabine is from about 100 mg to about 200 mg cumulative per first dosing cycle. In some embodiments, the second, reduced amount of decitabine is from about 70 mg to about 150 mg cumulative per subsequent dosing cycle. In some embodiments, the first amount of cedazuridine is from about 300 mg to about 700 mg cumulative per first dosing cycle. In some embodiments, the second, reduced amount of cedazuridine is from about 100 mg to about 500 mg cumulative per subsequent dosing cycle.

In some embodiments of the invention, the cancer treated with decitabine (and/or another hypomethylating agent) and cedazuridine is a hematological cancer, for example, a hematological cancer such as MDS, leukemia, or lymphoma. In particular embodiments, the leukemia is acute lymphocytic leukemia, acute myelogenous leukemia, chronic myelogenous leukemia, myeloproliferative neoplasms, or chronic myelomonocytic leukemia.

In some embodiments of the invention, the response of the cancer is measured by counting the number of leukemic blasts in the subject's bone marrow. In such cases, in some embodiments, the predetermined criterion is a blast count below a predefined percentage (e.g., a blast count of less than 5%).

In some embodiments of the invention, the response of the cancer is measured by measuring the DNA methylation in the subject.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations, and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

“Effective amount” refers to the amount required to produce a desired effect (e.g., treating cancer in a subject).

“Pharmaceutically acceptable” refers to those properties and/or substances that are acceptable to the patient from a pharmacological and/or toxicological point of view, and/or to the manufacturing pharmaceutical chemist from a physical and/or chemical point of view regarding composition, formulation, stability, patient acceptance, bioavailability and compatibility with other ingredients.

“Pharmaceutically acceptable excipient” can mean any substance, not itself a therapeutic agent, used as a carrier, diluent, adjuvant, binder, and/or vehicle for delivery of a therapeutic agent to a subject, or added to a pharmaceutical composition to improve its handling or storage properties or to permit or facilitate formation of a compound or composition into a unit dosage form for administration. Pharmaceutically acceptable excipients are well known in the pharmaceutical arts and are described, for example, in Remington's Pharmaceutical Sciences, Mack Publishing Co., Easton, Pa (e.g., 20th Ed., 2000), and Handbook of Pharmaceutical Excipients, American Pharmaceutical Association, Washington, D.C., (e.g., 1st, 2nd and 3rd Eds., 1986, 1994 and 2000, respectively). As will be known to those skilled in the art, excipients may provide a variety of functions and may be described as wetting agents, buffering agents, suspending agents, lubricating agents, emulsifiers, disintegrants, absorbents, preservatives, surfactants, colorants, flavorants, and sweeteners. Examples of pharmaceutically acceptable excipients include without limitation: (1) sugars, such as lactose, glucose and sucrose; (2) starches, such as corn starch and potato starch; (3) cellulose and its derivatives, such as sodium carboxymethyl cellulose, ethyl cellulose, cellulose acetate, hydroxypropylmethylcellulose, and hydroxypropylcellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically acceptable salt” refers to an acid or base salt of a compound of the invention, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quaternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

“Unit dosage form” refers to a physically discrete unit suitable as a unitary dosage for human or other animal subjects. Each unit dosage form may contain a predetermined amount of an active substance (e.g., decitabine and/or cedazuridine) calculated to produce a desired effect.

“Optional” or “optionally” means that the subsequently described event or circumstance may or may not occur, and that the description includes instances where the event or circumstance occurs and instances in which it does not.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

“Subject” refers to a cell or tissue, in vitro or in vivo, an animal or a human. An animal or human subject may also be referred to as a “patient.”

“Animal” refers to a living organism having sensation and the power of voluntary movement, and which requires for its existence oxygen and organic food.

“Mammal” refers to a warm-blooded vertebrate animal with hair or fur. Examples include without limitation members of the human, equine, porcine, bovine, murine, canine or feline species.

By the term “treat,” “treating,” or “treatment of” (or grammatically equivalent terms) it is meant that the severity of the subject's condition is reduced or at least partially improved or ameliorated and/or that some alleviation, mitigation or decrease in at least one clinical symptom is achieved. “Treating” in reference to a disease, disorder or condition may refer to: (i) inhibiting a disease, disorder, or condition, e.g., arresting its development; and/or (ii) relieving a disease, disorder or condition, e.g., causing regression of the clinical symptoms.

### Methods of Treating Cancer

Provided according to embodiments of the invention are methods of treating cancer in a subject in need thereof that include administering to the subject a first amount of decitabine (and/or another hypomethylating agent) and a first amount of cedazuridine in a first dosing cycle; measuring a response of the cancer to the first dosing cycle; and administering a second, reduced amount of decitabine (and/or another hypomethylating agent) and a second, reduced amount of cedazuridine in subsequent dosing cycle(s) if the response of the cancer to the first dosing cycle meets a predetermined criteria. A “hypomethylating agent,” as used herein (also known as DNA methyltransferase or DNMT inhibitors) refers to an administered drug that inhibits or reduces DNA methylation. Examples of hypomethylating agents include, but are not limited to, decitabine, cytidine, azacitidine (5-azacytidine), and guadecitabine. The invention will be discussed with reference to decitabine hereafter, but it should be understood that other hypomethylating agents may be used in combination with or in lieu of decitabine in the methods of the invention.

Administration of the decitabine and the cedazuridine (which includes administering pharmaceutical compositions that comprise decitabine and/or cedazuridine) to the subject may be performed via any accepted mode known to one skilled in the art, for example, orally, parenterally, by inhalation spray, topically, rectally, nasally, buccally, vaginally, intraocularly, intrapulmonarily, or via an implanted reservoir. The term “parenterally” includes without limitation subcutaneously, intravenously, intramuscularly, intraperitoneally, intrathecally, intraventricularly, intrasternally, intracranially, intratumorally, by injection into a blood vessel feeding a tumor, by intraosseous injection and by infusion techniques. In particular embodiments, the decitabine and/or cedazuridine are administered orally, such as in a combined solid oral dosage form.

Administering decitabine may be performed prior to, at substantially the same time with, or after cedazuridine. In some embodiments of the methods of the present invention, the administering of decitabine is concurrent with the administering of cedazuridine. The term “concurrent” as used herein encompasses wherein the administering of decitabine may be immediately followed by the administering of cedazuridine, and/or wherein the administering cedazuridine may be immediately followed by the administering of decitabine (e.g., at substantially the same time, e.g., less than 30 minutes, e.g., less than 30, 25, 20, 15, 10, 5 or less minutes apart). The term “concurrent” as used herein also encompasses wherein the administering of decitabine and cedazuridine is performed together, e.g., concomitant, e.g., in a single (e.g., combined) composition. In some embodiments, the administering of decitabine may be performed prior to the administering of cedazuridine (e.g., within 1, 2, 3, 4, 5, 6, 7, 8, 9, 10, 11, or 12 hrs). In some embodiments, the administering of cedazuridine is performed prior to the administering of decitabine (e.g., within 1, 2, 3, 4, 5, 6, 7, 8, 8, 9, 10, 11, or 12 hrs).

In some embodiments of the invention, the first dosing cycle and the subsequent dosing cycle(s) have a length of about 25 to about 30 days (e.g., 28 days). In some embodiments, in the first dosing cycle, the administering of decitabine and cedazuridine may be performed about 3 days to about 7 days per cycle (e.g., about 3, 4, 5, 6, or 7 days per cycle). As used herein, the “first dosing cycle” may not be the first dose of decitabine and/or cedazuridine that has been administered to the subject. The term “first” is used only to indicate that the dosing cycle occurs prior to the administering of the subsequent dosing cycle(s). In some embodiments, in the subsequent dosing cycle(s), the administering of decitabine and cedazuridine may be performed about 1 days to about 6 days per cycle (e.g., about 1, 2, 3, 4, 5, or 6 days per cycle). In both the first dosing cycle and the subsequent dosing cycle(s), the decitabine and/or cedazuridine may be administered in one dose per day or the daily dose may be split into multiple discrete doses that are administered at different times during the day. The subsequent dosing cycle(s) may include 1, 2, 3, 4, 5 or more subsequent dosing cycles. The dosage administered in each subsequent dosing cycle may be decreased based on the response of the cancer to the prior dosing cycle(s). In some cases, after the dosage has been decreased in at least one subsequent dosing cycle, the dosage may be further decreased if an additional criterion has been met, or in some cases, the dosage may be increased, for example, if the initial predetermined criterion is no longer met and/or another criterion is met. In some cases, increase or decrease of the dosage (or cessation of treatment) in subsequent cycles may be performed for another medical reason, such as neutropenia, an infection in the subject, etc.

For both the first dosing cycle and subsequent dosing cycle(s), in some embodiments, administering of decitabine and cedazuridine may be performed on consecutive days per cycle. For example, decitabine and cedazuridine may be administered on any 2 consecutive days. In some embodiments, decitabine and cedazuridine may be administered on any 3 consecutive days (e.g., on a Monday, Tuesday, and a Wednesday “MTW”; on a Tuesday, Wednesday, and a Thursday “TWTh”; on a Wednesday, Thursday, and a Friday “WThF”; on a Thursday, Friday, and Saturday “ThFS”; on a Friday, Saturday, and a Sunday “FSS”; on a Saturday, Sunday, and a Monday “SSM”; and/or on a Sunday, Monday, and a Tuesday “SMT”, etc.). In some embodiments, the administering of decitabine and cedazuridine may be performed on 4 consecutive days (e.g., MTWTh or any other combination of 4 consecutive days), the administering of decitabine and cedazuridine may be performed on 5 consecutive days (e.g., MTWThF or any other combination of 5 consecutive days), or on 6 consecutive days (e.g., MTWThFS or any other combination of 6 consecutive days), per cycle.

For both the first dosing cycle and subsequent dosing cycle(s), in some embodiments, the administering of decitabine and cedazuridine may be performed on non-consecutive days per cycle. Non-consecutive days may comprise a schedule of every-other day, (e.g., MWF), every two days, every three days, every four days, every five days, every six days, every seven days, etc. in a cycle. Non-consecutive days may comprise administering for a number of consecutive days (e.g., “on”), followed by a number of days without administering (e.g., “off”), followed by administering for a number of consecutive days (e.g., “on”), etc., within a cycle. For example, in some embodiments, the administering may be performed on 2 non-consecutive days (e.g., every Monday and Friday or any other combination of 2 non-consecutive days). In some embodiments, the administering may be performed on 3 non-consecutive days (e.g., MWF or any other combination of 3 non-consecutive days).

The reduced amount of decitabine and the reduced amount of cedazuridine administered in the subsequent dosing cycle(s) refers to a reduced cumulative amount per dosing cycle. Typically, the same amount of decitabine and cedazuridine are administered each day but in the subsequent dosing cycle(s), the decitabine and cedazuridine are administered for fewer days per cycle. However, in some embodiments, the decitabine and cedazuridine may be administered the same number of days in the subsequent cycle(s) as in the first dosing cycle but a reduced amount of the decitabine and a reduced amount of cedazuridine are administered each day.

In some embodiments, the amount of decitabine administered in the first dosing cycle is from about 100 mg to about 200 mg cumulative per first dosing cycle (e.g., about 100, 110, 120, 130, 140, 150, 160, 170, 180, 190, or 200 mg or any value or range therein, e.g., 175 mg). If the response of the cancer to the decitabine and cedazuridine administered meets the predetermined criterion, the amount of decitabine administered in at least one subsequent dosing cycle is reduced. For example, in some embodiments, the second, reduced amount of decitabine is from about 70 mg to about 150 mg cumulative per subsequent dosing cycle (e.g., about 70, 80, 90, 100, 110, 120, 130, 140, or 150 mg or any value or range therein, e.g., 105 mg).

In some embodiments, the amount of cedazuridine administered in the first dosing cycle is from about 300 mg to about 700 mg cumulative per first dosing cycle (e.g., about 300, 400, 500, 600, or 700 mg or any value or range therein, e.g., 500 mg). If the response of the cancer to the decitabine and cedazuridine meets the predetermined criterion, the amount of decitabine and the amount of cedazuridine administered in at least one subsequent cycle is reduced. In some embodiments, the second, reduced amount of cedazuridine is from about 100 mg to about 500 mg cumulative per subsequent dosing cycle (e.g., about 100, 200, 300, 400, or 500 mg or any value or range therein, e.g., 300 mg). The reduction in dosage in decitabine in the subsequent cycle(s) is typically proportional to the reduction in dosage of the cedazuridine, but in some embodiments, the decitabine may be reduced more than the cedazuridine in the subsequent cycle(s), or the cedazuridine may be reduced more than the decitabine in the subsequent cycle(s).

For both the first dosing cycle and subsequent dosing cycle(s), in some embodiments, the administration regimen may include pretreatment and/or co-administration with at least one additional therapeutic agent. In such case, the decitabine and cedazuridine and the at least one additional therapeutic agent may be administered concurrently, separately, or sequentially.

Examples of chemotherapeutic agents include without limitation: alkylating agents (e.g., which may include doxorubicin, cyclophosphamide, estramustine, carmustine, mitomycin, bleomycin and the like); antimetabolites (e.g., which may include 5-Fluoro-Uracil, capecitabine, gemcitabine, nelarabine, fludarabine, methotrexate and the like); platinating agents (e.g., which may include cisplatin, oxaliplatin, carboplatin and the like); topoisomerase inhibitors (e.g., which may include topotecan, irinotecan, etoposide and the like); tubulin agents (e.g., which may include paclitaxel, docetaxel, vinorelbine, vinblastine, vincristine, other taxanes, epothilones, and the like); signaling inhibitors (e.g., kinase inhibitors, antibodies, farnesyltransferase inhibitors, and the like); IDH1/2 inhibitors (e.g., enasidenib and ivosidenib), Flt-3 inhibitors (midostaurin and sorafenib), venetoclax, and other chemotherapeutic agents (e.g., tamoxifen, anti-mitotic agents such as polo-like kinase inhibitors or aurora kinase inhibitors, and the like).

Examples of administration regimens include, without limitation: administration of decitabine, cedazuridine, and/or the additional therapeutic agent in a sequential manner; and co-administration of decitabine and cedazuridine and/or additional therapeutic agent in a substantially simultaneous manner (e.g., as in a single unit dosage form), in multiple, separate unit dosage forms for each compound, or a single unit dosage form of decitabine and cedazuridine (e.g., a solid oral dosage form) and a single unit dosage of the additional therapeutic agent(s).

In some embodiments, a time period of 0 to 31 days or more (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more) may pass between multiple 28-day treatment cycles of the present invention. The time period of no treatment may be desirable to allow a subject (e.g., a human patient) of the present invention to have adequate health to continue treatment. The time period between treatment cycles can be determined by a physician using standard techniques in the art and may be determined individually on a per-subject basis, for example, as based on adequate blood count, e.g., adequate lack of neutropenia (e.g., absolute neutrophil count (ANC) in the subject of at least or greater than 0.5×10⁹ cells/L) and may be adjusted over the course of treatment based on the judgement of the administering physician. In some embodiments, the time period between treatment cycles may be minimal, e.g., no time period, e.g., immediately starting on the next 28-day time period. In some embodiments, the time period between treatment cycles may be 1 week, 2 weeks, 3 weeks, 4 weeks, 5 weeks, 6 weeks, or more.

As used herein, “cancer” refers to an abnormal growth of cells which tend to proliferate in an uncontrolled way and, in some cases, to metastasize (spread). In particular embodiments of the invention, the cancer is a hematologic cancer including, for example, blood cancers (e.g., such as myeloid leukemia [acute and chronic], acute lymphoblastic leukemia, chronic lymphocytic leukemia, myeloproliferative diseases, multiple myeloma, myelodysplastic syndrome and the like), Hodgkin's disease, and non-Hodgkin's lymphoma.

In some embodiments of the invention, the cancer that is treated is a hematological cancer selected from MDS, leukemia, and lymphoma. In particular embodiments, the leukemia is acute lymphocytic leukemia, acute myelogenous leukemia, chronic myelogenous leukemia, myeloproliferative neoplasms, or chronic myelomonocytic leukemia.

In some embodiments of the invention, the cancer is leukemia and the response of the cancer that is measured is the number of leukemic blasts in the subject's bone marrow, and the predetermined criterion is a predetermined concentration of leukemic blasts in the bone marrow. For example, in some embodiments, the predetermined criterion is met if the subject's leukemic blasts are less than 5%, less than 4%, less than 3%, less than 2% or less than 1%, or there are no detectable leukemic blasts.

In some embodiments, a reduction in DNA methylation is evidence that the dosing regimen is acting on the target. DNA methylation in the subject may be quantitatively and/or qualitatively evaluated by any standard technique in the art, e.g., as measured by a marker of relative global methylation as compared to a control, e.g., as measured by LINE-1 methylation as compared to a control. Exemplary methods of DNA methylation analysis include sequencing and array-based methods, including whole genome bisulfite sequencing, target bisulfite sequencing (e.g., TruSeq MethylCapture) and DNA methylation array, as well as long read DNA methylation sequencing. For example, in some embodiments, the predetermined criterion is met if there is a reduction in LINE-1 methylation in the subject by at least 5% (e.g., at least 5, 6, 7, 8, 9, 10, 11, 12, 13, 14, or 15% or more) as compared to a control measurement, e.g., as compared to LINE-1 methylation in the subject prior to the first dosing cycle (e.g., subject baseline LINE-1 methylation). For example, in some embodiments, the predetermined criterion is a reduction of LINE-1 methylation in the subject by at least 5%, at least 8%, at least 10% or at least 15% or more. In some embodiments, the predetermined criterion is a reduction of LINE-1 methylation in the subject by about 5% to about 20%, about 6% to about 15%, or by about 8% to about 10%.

Other measures of the response of the cancer to the decitabine and cedazuridine may be used. As used herein, the term “response” refers to a change in the level of the cancer in the subject, for example, a reduction in the amount of cancer in the subject. Reduction may be measured by serological markers (e.g., increase in size and/or number of leukemia cells, platelet count, biomarkers), immunohistochemistry, flow cytometry, biopsy, solid tumor burden, and imaging modalities including PET-CT, CT, and MRI. Exemplary biomarkers for lymphoma include, for example, cluster differentiation markers, CD20 or CD30. Response can be measured at any time during the first cycle, for example after the last day of treatment of the dosing cycle, for example, day 6, 7, 8, 9, 10, 11, 12, 13, 14, 15, 16, 17, 18, 19, 20, 21, 22, 23, 24, 25, 26, 27, or 28 of a first 28-day dosing cycle. In some embodiments, two or more criteria may need to be met to administer a reduced dosage of decitabine and/or cedazuridine in the subsequent cycles.

In particular embodiments of the invention, in the first dosing cycle, the decitabine and cedazuridine are administered on days 1-5 of a 28-day dosing cycle, for example, in a solid oral dosage form that includes both decitabine (and/or a pharmaceutically acceptable salt thereof) and cedazuridine (and/or a pharmaceutically acceptable salt thereof) and a pharmaceutically acceptable excipient. In some embodiments, the solid oral dosage form includes 35 mg of decitabine and 100 mg of cedazuridine. In some embodiments, if the subject's response to the cancer (e.g., leukemia) meets a predefined criterion (e.g., a leukemic blast count of less than 5% in the subject's bone marrow), in the subsequent dosing cycle(s), the decitabine and cedazuridine are administered on days 1-4, days 1-3, or days 1 and 2, of a 28-day dosing cycle, for example, by administering the same solid oral dosage form as in the first cycle (but administered on fewer days). In an embodiment, the methods may comprise administration of an additional therapeutic agent, e.g., chemotherapeutic agent.

### Pharmaceutical Compositions for Use in Methods of the Invention

In the methods of the invention, the decitabine (and/or another hypomethylating agent) and the cedazuridine may be administered in pharmaceutical compositions. In some embodiments, a pharmaceutical composition may include an effective amount of decitabine, and/or a pharmaceutically acceptable salt thereof, and a pharmaceutically acceptable excipient. In some embodiments, a pharmaceutical composition may include an effective amount of cedazuridine, and/or a pharmaceutically acceptable salt thereof, and a pharmaceutically acceptable excipient. In some embodiments, a pharmaceutical composition may include an effective amount of decitabine, and/or a pharmaceutically acceptable salt thereof, an effective amount of cedazuridine, and/or a pharmaceutically acceptable salt thereof, and a pharmaceutically acceptable excipient.

The compositions including decitabine and/or cedazuridine may be formulated for administration in solid or liquid form, including those adapted for the following: (1) oral administration, for example, drenches (for example, aqueous or non-aqueous solutions or suspensions), tablets (for example, those targeted for buccal, sublingual and systemic absorption), caplets, boluses, powders, granules, pastes for application to the tongue, hard gelatin capsules, soft gelatin capsules, mouth sprays, troches, lozenges, pellets, syrups, suspensions, elixirs, liquids, emulsions and microemulsions; (2) parenteral administration, for example, by subcutaneous, intramuscular, intravenous or epidural injection as, for example, a sterile solution or suspension; (3) topical application, for example, as a cream, ointment, patch, pad or spray applied to the skin; (4) intravaginally or intrarectally, for example, as a pessary, cream or foam; (5) sublingually; (6) ocularly; (7) transdermally; or (8) nasally. The pharmaceutical compositions may be formulated for immediate, sustained, or controlled release.

In some embodiments, the pharmaceutical compositions are formulated for oral administration. In further embodiments, the pharmaceutical compositions are formulated for oral administration in solid form.

Pharmaceutical compositions of the invention can be prepared using known materials and techniques, which may include but are not limited to mixing and/or blending the compound of the invention with the pharmaceutically acceptable excipient and optional therapeutic agent(s).

The methods of the invention may be performed with a unit dosage form and/or a kit comprising at least one unit dosage form, which unit dosage form comprises decitabine and/or cedazuridine, and/or a pharmaceutical composition described herein. In some embodiments, the unit dosage form comprises about 5, 10, 15, 25, 20, 30, or 35 mg decitabine. For example, in some embodiments, the unit dosage form of decitabine may be about 5 mg to about 35 mg, about 5 mg to about 30 mg, about 5 mg to about 25 mg, about 5 mg to about 20 mg, about 5 mg to about 15 mg, about 5 mg to about 10 mg, about 7 mg to about 20 mg, or about 5 mg, about 10 mg, about 14, about 15 mg, about 21, about 28 mg, or about 35 mg. In some embodiments, the unit dosage form of cedazuridine comprises about 40 mg to about 700 mg cedazuridine. For example, in some embodiments, the unit dosage form of cedazuridine may comprise about 40 mg to about 150 mg, about 40 mg, about 60, about 75 mg, about 80, about 100 mg, about 150 mg, about 200 mg, or about 250 mg cedazuridine.

In some embodiments of the invention, the solid oral dosage form is a unit dosage form that comprises about 35 mg decitabine and about 100 mg of cedazuridine. In some embodiments of the invention, the unit dosage form comprises about 35 mg decitabine and about 100 mg of cedazuridine and at least one pharmaceutically acceptable excipient. In some embodiments, the solid oral dosage form is a unit dosage form that comprises about 14 mg, about 21 mg, or about 28 mg of decitabine; and about 40 mg, about 60 mg, or about 80 mg of cedazuridine, optionally with at least one pharmaceutically acceptable excipient.

The kit may further comprise a container and/or a package suitable for commercial sale. The container can be in any conventional shape or form as known in the art which is made of a pharmaceutically acceptable material, such as a paper or cardboard box, a glass or plastic bottle or jar, a re-sealable bag, or a blister pack with individual dosages for pressing out of the pack according to a therapeutic schedule. More than one container can be used together in a single package. For example, tablets may be contained in a blister pack which is in turn contained within a box.

The kit may further comprise information. The information may be provided on a readable medium. The readable medium may comprise a label. The information may be directed towards a physician, pharmacist, or patient. The information may indicate that the unit dosage form may cause one or more adverse effects. The information may comprise instructions for administering the unit dosage form, such as in a manner described herein. These instructions may be provided in a variety of ways. For example, the information may include a table including a variety of weights or weight ranges and appropriate dosages for each weight or weight range.

The information can be associated with the container, for example, by being: written on a label (e.g., the prescription label or a separate label) adhesively affixed to a container; included inside a container as a written package insert; applied directly to the container such as being printed on the wall of a box or blister pack; or attached as by being tied or taped, for example as an instructional card affixed to the neck of a bottle via a string, cord or other line, lanyard or tether type device.

It will be apparent to those skilled in the art that specific embodiments of the present invention may be directed to one, some or all of the above-indicated aspects as well as other aspects, and may encompass one, some or all of the above- and below-indicated embodiments, as well as other embodiments.

1. A method of treating cancer in a subject in need thereof, comprising:
(i) administering to the subject a first amount of a hypomethylating agent (e.g., decitabine) and a first amount of cedazuridine in a first dosing cycle;
(ii) measuring a response of the cancer to the first dosing cycle; and
(iii) administering a second, reduced amount of the hypomethylating agent and a second, reduced amount of the cedazuridine in subsequent dosing cycle(s) if the response of the cancer measured in (ii) meets a predetermined criterion.

2. The method of claim 1, wherein the cancer is a hematological cancer.

3. The method of claim 2, wherein the hematological cancer is selected from myelodysplastic syndrome (MDS), leukemia, and lymphoma.

4. The method of claim 3, wherein the leukemia is acute lymphocytic leukemia, acute myelogenous leukemia, chronic myelogenous leukemia, myeloproliferative neoplasms, or chronic myelomonocytic leukemia.

5. The method of claim 1, wherein the cedazuridine is administered concurrently with the hypomethylating agent in step (i) and in step (iii).

6. The method of claim 5, wherein the hypomethylating agent (e.g., decitabine) and cedazuridine are administered as a single composition (e.g., a single solid oral dose form) in step (i) and step (iii).

7. The method of claim 1, wherein the first dosing cycle and the subsequent dosing cycle(s) each independently have a length of about 25 to about 30 days (e.g., 28 days).

8. The method of claim 7, wherein in step (i), the administering is performed on about 3 days to about 7 days (e.g., about 5 days) in the first dosing cycle.

9. The method of claim 1, wherein the administering is performed on fewer days in the subsequent dosing cycle(s) in step (iii) than in the first dosing cycle in step (i).

10. The method of claim 9, wherein the administering is performed on about 1 day to about 6 days per subsequent dosing cycle (e.g., about 1, 2, 3, or 4 days) in step (iii).

11. The method of claim 1, wherein the hypomethylating agent is decitabine and the first amount of decitabine is from about 100 mg to about 200 mg cumulative per first dosing cycle (e.g., about 100, 110, 120, 130, 140, 150, 160, 170, 180, 190, or 200 mg or any value or range therein, e.g., 175 mg).

12. The method of claim 11, wherein the second, reduced amount of decitabine is from about 70 mg to about 150 mg cumulative per subsequent dosing cycle (e.g., about 70, 80, 90, 100, 110, 120, 130, 140, or 150 mg or any value or range therein, e.g., 105 mg).

13. The method of claim 1, wherein the first amount of cedazuridine is from about 300 mg to about 700 mg cumulative per first dosing cycle (e.g., about 300, 400, 500, 600, or 700 mg or any value or range therein, e.g., 500 mg).

14. The method of claim 1, wherein the second, reduced amount of cedazuridine is from about 100 mg to about 500 mg cumulative per subsequent dosing cycle (e.g., about 100, 200, 300, 400, or 500 mg or any value or range therein, e.g., 300).

15. The method of claim 1, wherein the cancer is leukemia and measuring the response of the cancer in step (ii) comprises counting the number of leukemic blasts in the subject's bone marrow and the predetermined criterion is a leukemic blast count below a predefined percentage.

16. The method of claim 15, wherein the predetermined criterion is a leukemic blast count of below 5% (e.g., below 4%, below 3%, below 2%, or below 1%).

17. The method of claim 1, wherein the hypomethylating agent and the cedazuridine are administered intravenously, orally, and/or subcutaneously (e.g., orally in a solid dose form).

18. The method of claim 1, further comprising administering an additional therapeutic agent.

19. The method of claim 1, wherein the subject is a mammal.

20. The method of claim 19, wherein the subject is a human.
